(19) United States
(12) Patent Application Publication
(10) Pub. No.: US 2013/0331332 A1
(43) Pub. Date: Dec. 12, 2013

(54) COMPOSITION COMPRISING PEROXISOME PROLIFERATOR-ACTIVATED RECEPTOR-GAMMA

(71) Applicants: Heiko Barg, Weinheim (DE); Rainer Pooth, Bad Soden/Taunus (DE)

(72) Inventors: Heiko Barg, Weinheim (DE); Rainer Pooth, Bad Soden/Taunus (DE)

(73) Assignee: MERZ PHARMA GmbH & Co. KGaA, Frankfurt am Main (DE)

(21) Appl. No.: 14/001,748

(22) PCT Filed: Feb. 29, 2012
(86) PCT No.: PCT/EP2012/053414

§ 371 Date: Aug. 27, 2013

Related U.S. Application Data
(60) Provisional application No. 61/447,726, filed on Mar. 1, 2011.

(30) Foreign Application Priority Data
Mar. 1, 2011 (EP) 11156450.6

(51) Int. Cl. A61K 8/64; A61Q 19/00; A61Q 19/08

(52) U.S. Cl. CPC A61K 8/64; A61Q 19/08; A61Q 19/00
USPC 514/18.8; 530/350

(57) ABSTRACT

An injectable composition comprising peroxisome proliferator-activated receptor-gamma for subcutaneous administration and a method for improving imperfections of the skin, wherein the injectable composition is subcutaneously administered at the area of skin imperfections comprising the steps: a) identifying an area of skin imperfections, b) administering a safe and cosmetically effective amount of the composition subcutaneously to the area of skin imperfections.

### FIELD OF INVENTION

The present invention relates to an injectable composition comprising peroxisome proliferator-activated receptor-gamma for subcutaneous administration. The present invention also relates to the use of the composition for improving imperfections of the skin. Further, the present invention relates to a method for improving imperfections of the skin, wherein an injectable composition comprising peroxisome proliferator-activated receptor-gamma is subcutaneously administered at the area of skin imperfections comprising the steps: a) identifying an area of skin imperfections, b) administering a safe and cosmetically effective amount of the composition subcutaneously to the area of skin imperfections.

### BACKGROUND OF THE INVENTION

The skin is a complex tissue which is generally divided into three main layers, the epidermis, the dermis, and underneath the dermis the hypodermis. This deepest layer of the skin contains adipose cells and is also known as the subcutaneous fat layer. The skin is an anatomical barrier to the environment performing different objects one being the controlling the body temperature. The skin changes with age. The subcutaneous fat layer, which provides insulation and padding, thins and loses fat. This increases the risk of skin injury and reduces the ability to maintain body temperature. Particularly in the face and neck the amount of fat in the adipose tissue reduces with age. As a result of this the cutis is much less well padded which is associated with the formation of visible skin wrinkles, laugh and smile lines, sunken cheeks, and facial creases especially over the areas where muscle movements occurs. Besides the visceral local fat relocation with age there can be a substantial local lipodystrophy associated with virus infection like Acquired Immune Deficiency Syndrome (AIDS).

The changes of skin are among the most visible signs of aging. Most notable changes of the skin are the appearance of wrinkles and sagging skin. However, an increasing number of people still feel young and do not want to look old. Many products like creams and lotions claim to reduce wrinkles but often as a marketing argument only and without substantiated effect. Alternatives for treating skin wrinkles include the injection of fillers into the target area. Dermal fillers include collagen, hyaluronic acid, aliginate, and also cells for example human dermal fibroblasts, minced fat tissue, or autologous transplanted cells or tissues.

Such fillers address the volume deficiencies and cause always foreign body reactions that can lead to inflammatory nodules, granuloma several months up to years post injection. In general filler bear also the risk for migration. Dermal fillers either are non-permanent fillers and therefore resorbable or are permanent and non-resorbable in their effect. However, synthetic or animal derived filler material may cause hypersensitivity reactions. Autologous fat injections taking a patient's fat from one location and transferring it in the same patient to another location are still very complex, expensive and invasive treatment connected with considerable downtime i. e. not patient convenient.

Thus, there is an ongoing demand for improvements in improving imperfections of the skin.

Therefore, it is an object of the present invention to provide an injectable composition exhibiting a good efficiency in regenerating fatty tissue.

### SUMMARY OF THE INVENTION

Surprisingly, it was found that an injectable composition comprising peroxisome proliferator-activated receptor-gamma for subcutaneous administration meets the object of the present invention.

Therefore, in one aspect, the present invention provides an injectable composition comprising peroxisome proliferator-activated receptor-gamma for subcutaneous administration. The subcutaneous administration of peroxisome proliferator-activated receptor-gamma results in an improvement of skin imperfections, e. g. due to aging or virus infection. The injectable composition can be a cosmetic composition or a medical composition.

The invention also relates to an injectable composition comprising peroxisome proliferator-activated receptor-gamma for subcutaneous administration, wherein the composition comprises: a) peroxisome proliferator-activated receptor-gamma, b) a pharmaceutically acceptable carrier, and optionally c) a dermal filling material. The injectable composition can be a cosmetic composition or a medical composition.

This invention also relates to the use of an injectable composition according to the present invention for subcutaneous administration for improving imperfections of the skin, for use in facial or body contouring, in facial or body shaping, as face or body filler, or for the treatment of large area volume deficiencies, and the use as dermal filler. The injectable composition to be used can be a cosmetic composition or a medical composition. Hence, the invention can relate to a non-therapeutic or therapeutic use of the injectable composition.

This invention further relates to a method for improving imperfections of the skin, wherein an injectable composition according to the present invention is subcutaneously administered at the area of skin imperfections comprising the steps: a) identifying an area of skin imperfections, b) administering a safe and cosmetically effective amount of the composition subcutaneously or dermal to the area of skin imperfections. The injectable composition can be a cosmetic composition or a medical composition. Hence, the invention can relate to a non-therapeutic or therapeutic method. The subcutaneous administration of the composition comprising peroxisome proliferator-activated receptor-gamma results in a reduction of the appearance of skin imperfections around the identified area of skin imperfections.

The injectable composition of this invention may further contain at least one agonist of the peroxisome proliferator-activated receptor-gamma. The injectable composition of this invention may further contain retinoic acid, retinol, retinal and/or retinoid X receptor.

The injectable composition of this invention may further contain a dermal filling material including, but not limited to, collagen, cross-linked collagen, hyaluronic acid, crosslinked hyaluronic acid, poly lactic acid, calcium hydroxyl apatite, chondroitin sulfate, polyesters, polyethylene glycols, polycarbonates, polyvinyl alcohols, polyacrylamides, polyamides, polyacrylates, polyetheresters, polymethacrylates, polyurethanes, polycaprolactone, polyphophazenes, polyorthoesters, polyglycolides, copolymers of lysine and lactic acid, copolymers of lysine-RGD and lactic acid, chitosan, alginates, pectin, gelatin, gellan, carrageenan, cells, stem cells, adult stem cells, embryonic stem cells, induced pluripotent cells, progenitor cells, minced tissues, autologous transplanted cells, fat or tissues, and mixtures thereof.

Other features and advantages of the present invention will be apparent from the detailed description of the invention and from the claims.

### DETAILED DESCRIPTION OF THE INVENTION

According to the invention an injectable composition comprising peroxisome proliferator-activated receptor-gamma is provided for subcutaneous administration. The composition of the present invention shows a good bioavailability and was found to induce the sustainable (re)-generation of natural fat without any strong invasive techniques. Apart from that, it is able to reduce the side effects compared to known fillers. Further, it can lead to a natural look. The composition of the present invention can be used as a filler or body contourer. Moreover, a consistent tolerability and safety are achieved by the fact that a natural compound is used in contrast to synthetic or animal derived filler material. Therefore, hypersensitivity reactions are unlikely. Further, patient convenient and a more causative treatment can be provided.

It is thought that the advantageous effects are derived due to the ability of the peroxisome proliferator-activated receptor-gamma to interact with the adipocytes. Peroxisome proliferator-activated receptor gamma (PPAR-gamma) belongs to the nuclear hormone receptor subfamily of transcription factors and is thought to up-regulate the adipocyte tissue. This ability to up-regulate the adipocyte tissue could be used to improve the amount of fat in the subcutaneous fat tissue. In contrast to topic delivery, the subcutaneous administration can provide efficacious delivery to the site of action. Further, the composition of the present invention can provide a long-term effect even in the treatment of large area volume deficiencies as body's own subdermal fat is induced for regeneration.

As used herein, the term “injectable” means that the composition of the present invention can be injected into a target area of the body of a living subject such as mammals, using any injection means including, but not limited to, needles, microneedles, syringes, and the like. The composition can be administered by a minimal-invasive injection. Administration by a minimal-invasive injection can induce a sustainable (re)-generation of natural fat without any strong invasive techniques.

The term “subcutaneous administration” means directly depositing in or underneath the skin, or in the subcutaneous fat layer. Usable application means are needles, microneedles, multi-needle arrays, syringes, or similar devices, e. g. injection jet.

The term “subcutaneous adipose tissue” refers to tissue in a layer that lies below the dermis of vertebrate skin, also called hypodermis.

The term “imperfection” refers to loss or absence of perfection. More specifically, “imperfections of skin” refer to conditions, defects or flaw in the skin diminishing the appearance, e. g. lipodystrophy, driven by aging or virus infection. Examples of “imperfections of skin” are wrinkles, slack eyelids, crow's feet, nasolabial wrinkles, scarred or furrowed skin, sagged skin and skin indentations. Facial imperfections of skin include, but are not limited to, frown lines, glabellar lines, nasolabial folds, forehead wrinkles, anger wrinkles, worry wrinkles, crow's feet or periorbital lines, smile lines, vertical or perioral lip lines, marionette lines or oral commissures, acne scars, cheek depressions, facial scars, lips and the like.

The term “skin defect” includes, but is not limited, to wrinkled skin, scarred or furrowed skin, folding skin, sagging skin. Further, conditions or defects of the skin are not limited to the aging skin but also include conditions displaying the appearance of an aesthetic deficiency like acne, or other irregularities of the skin, skin indentations after liposuction of cellulite or other skin areas, effects secondary to skin grafting or other surgically-induced irregularities.

The term “contouring” means adjusting, shaping, reforming or changing the features to a more youthful, full, and healthy look and to ameliorate the appearance of skin defects. This includes volume deficiencies all over the body to be addressed for rejuvenation or beautification in younger subjects e. g. upper arms, breast, buttocks. The term skin imperfection refers also to large area volume deficiencies in hands, décolletage, etc.

The term “pharmaceutically acceptable” means that the respective compounds or carriers are suitable for subcutaneous administration without undue toxicity, incompatibility, instability, irritation, allergic response, and the like. This term is not intended to limit the use of the compound or the product which it describes as a pharmaceutical, but to indicate the compatibility to the subject.

The term “cosmetically effective amount” means an amount of a compound or composition sufficient for treating an skin imperfection or facial contouring, but low enough to avoid serious side effects.

Unless defined otherwise, the technical and scientific terms used herein have the same meaning as commonly understood by one of ordinary skill in the art to which the invention belongs.

In one embodiment, the peroxisome proliferator-activated receptor-gamma has a concentration in the range from about 0.00001% by weight to about 5% by weight, based on the total weight of the composition. In another embodiment, the peroxisome proliferator-activated receptor-gamma has a concentration in the range from about 0.001% to about 1% by weight, based on the total weight of the composition. In a further embodiment, the peroxisome proliferator-activated receptor-gamma has a concentration in the range from about 0.01% to about 0.1% by weight, based on the total weight of the composition.

Peroxisome proliferator-activated receptor-gamma can be produced recombinant by biotechnological means e.g. expression in known hosts like Escherichia coli (E. coli), Bacillus megaterium, Bacillus subtilis, yeast, or Chinese hamster ovary (CHO) cells. Also, fungal hosts such as Aspergillus niger, Aspergillus nidulans and Pichia pastoris can be used. The protein can for example be cleaned up by affinity chromatography. Alternatively, peroxisome proliferator-activated receptor-gamma can be produced by chemical synthesis. Further, peroxisome proliferator-activated receptor-gamma is commercially available.

According to an embodiment of the present invention, the composition can comprise at least one agonist of the peroxisome proliferator-activated receptor-gamma selected from the group consisting of eicosanoids especially prostaglandins such as Δ12-prostaglandin J2 and 15-deoxy-Δ12-prostaglandin J2, thiazoliden derivatives such as rosiglitazone, ciglitazone, troglitazone, englitazone and pioglitazone, non steroidal anti-inflammatory drugs (NSAID), unsatturated fatty acids, alpha-linoleic acid, arachidonic acid, docosahexaenoic acid, eicosapentaenoic acid, biphenyl derivatives, N-(phenyloxazol-4-yl-methoxymethyl)-cyclohexyl-succinic acid amide derivatives, and mixtures thereof.

As used herein, the term “agonist of the peroxisome proliferator-activated receptor-gamma” refers to a compound that directly interacts with the peroxisome proliferator-activated receptor-gamma protein, and stimulates its interaction with retinoid X receptors and/or its target genes, to produce a physiological effect. By interaction with peroxisome proliferator-activated receptor-gamma the agonist can enhance and/or prolong the effect of the composition comprising peroxisome proliferator-activated receptor-gamma.

In an embodiment of the invention the thiazoliden derivative is rosiglitazone, (RS)-5-((4-(2-(methyl-2-pyridinylamino)ethoxy)phenyl)methyl)-2,4-thiazolidinedione. A further example of a thiazoliden derivative that can be comprised in the composition according to the present invention is troglitazone, (RS)-5-(4-(6-hydroxy-2,5,7,8-tetramethylchroman-2-yl-methoxy)benzyl)-2,4-thiazolidinedione). In a further embodiment of the invention the thiazoliden derivative is pioglitazone, (RS)-5-{p-[2-(5-ethyl-2-pyridyl)ethoxy]benzyl}-2,4-thiazolidinedione.

In one embodiment, the agonist of the peroxisome proliferator-activated receptor-gamma has a concentration in the range from about 0.00001% by weight to about 5% by weight, based on the total weight of the composition. In another embodiment, the agonist of the peroxisome proliferator-activated receptor-gamma has a concentration in the range from about 0.001% by weight to about 1% by weight, based on the total weight of the composition. In a further embodiment, the agonist of the peroxisome proliferator-activated receptor-gamma has a concentration in the range from about 0.01% by weight to about 0.1% by weight, based on the total weight of the composition.

According to another embodiment of the present invention, the composition can comprise retinoic acid, retinol, retinal and/or retinoid X receptor. Retinoid X receptor forms a heterodimer with peroxisome proliferator-activated receptor-gamma. By interaction with retinoid X receptor the effect of the composition comprising peroxisome proliferator-activated receptor-gamma can be enhanced. In one embodiment, the retinoic acid, retinol, retinal or retinoid X receptor has a concentration in the range from about 0.00001% by weight to about 5% by weight, based on the total weight of the composition. In another embodiment, retinoic acid, retinol, retinal or retinoid X receptor has a concentration in the range from about 0.001% by weight to about 1% by weight, based on the total weight of the composition. In a further embodiment, retinoic acid, retinol, retinal or retinoid X receptor has a concentration in the range from about 0.01 by weight % to about 0.1% by weight, based on the total weight of the composition.

According to an embodiment of the present invention, the composition further comprises a dermal filling material selected from the group consisting of collagen, cross-linked collagen, hyaluronic acid, crosslinked hyaluronic acid, poly lactic acid, calcium hydroxyl apatite, chondroitin sulfate, polyesters, polyethylene glycols, polycarbonates, polyvinyl alcohols, polyacrylamides, polyamides, polyacrylates, polyetheresters, polymethacrylates, polyurethanes, polycaprolactone, polyphophazenes, polyorthoesters, polyglycolides, copolymers of lysine and lactic acid, copolymers of lysine-RGD and lactic acid, chitosan, alginates, pectin, gelatin, gellan, carrageenan, cells, stem cells, adult stem cells, embryonic stem cells, induced pluripotent cells, progenitor cells, minced tissues, autologous transplanted cells, fat or tissues, and mixtures thereof.

As used herein, the term “dermal filling material” refers to a material that is used for cosmetic and aesthetic needs to address volume deficiencies. In one embodiment, the dermal filling material can be collagen. In another embodiment, the dermal filling material can be cross-linked collagen wherein the collagen is cross-linked with one or more sugars. In alternative embodiments, the dermal filling material is alginate. The term “alginate” refers to a naturally occurring anionic unbranched polysaccharide which is isolated from marine brown algae. It is built up from homopolymeric groups of -D-mannuronic acid and -L-guluronic acid, separated by heteropolymeric regions of the two acids. A highly pure alginate which is usable according to an embodiment of the present invention can be isolated by using homogeneous algal raw material and standardized processes. The biocompatibility requirements are thereby met. In a further embodiment, the dermal filling material is cross-linked alginate, for example barium-cross-linked alginate. Alginates crosslinked with barium form stable hydrogels. In a further embodiment, the dermal filling material can be cells, for example human dermal fibroblasts.

In one embodiment, the dermal filling material is hyaluronic acid. Hyaluronic acid as dermal filling material can constitute from about 0.001% by weight to about 8% by weight of the final composition. In another embodiment, the dermal filling material hyaluronic acid can constitute about 1% by weight to about 4% by weight of the final composition. In alternative embodiments, the dermal filling material hyaluronic acid can constitute about 2% by weight to about 2.5% by weight of the final composition.

According to an embodiment of the present invention the composition is a subcutaneous injection. Examples for subcutaneous injection include aqueous solutions, suspensions, oily solutions, emulsions, microemulsions, liposomes, microspheres, nanoparticles and implants. The advantage of subcutaneous injections is the rapid onset of action and that the effect is restricted to the targeted tissue. Furthermore, the systemic availability of compounds over time is reduced, since drug absorption from subcutaneous tissue is slow.

According to the invention the composition may comprise a medium in which the peroxisome proliferator-activated receptor-gamma is suspended. Said medium may be sterile water, phosphate-buffered saline (PBS), ringer solution, isotonic saline solution (0.9%), trometamol, citrate, carbonate, acetate, borate, amino acid, diethylamine, glucono delta lactone, glycine, lactate, maleic acid, methanesulfonic acid, monoethanolamine, tartrate buffer of choice or any combination thereof.

The present invention can further comprise one or more excipients selected from antioxidants, buffers, tonicity agents, hydrating agents, viscosity enhancers and/or viscosity modifiers, surface active agents, complex builder, anti-foaming agent, preservative or a mixture thereof. Examples for such additive substances like complex builders, anti-foaming agents, and/or preservatives are ethylenediaminetetraacetic acid (EDTA), cresol and its derivatives, benzoic acid, 4-hydroxybenzoic acid ester, and/or sorbic acid. Exemplary surfactants include nonionic surfactants such as polysorbates such as polysorbate 20 or polysorbate 80, cetrimoniumbromid, cetylpyridiniumchlorid, deoxycholate, or mixtures thereof.

Antioxidants may be, but are not limited to, vitamin E, vitamin C, glutathione, coenzyme Q, resveratrol, quercetin, bisulfite sodium, butylated hydroxyl anisole/toluene, cysteinate, dithionite sodium, gentisic acid, glutamate, formaldehyde sulfoxylate sodium, metabisulfite sodium, monothiogylcerol, propyl gallate, sulfite sodium, thiogycolate sodium, flavonoids, catalase, lycopene, carotenes, lutein, superoxide dismutase and peroxidises, Zinc or mixtures thereof.

The composition as claimed in the instant invention may further comprise one or more active pharmaceutical ingredients selected from the group of anesthetics, analgenics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

The term “active pharmaceutical ingredient” refers to all structures, which are pharmacologically active, thus resulting in a pharmacological effect in mammal and all known chemical forms thereof. Examples are, but not limited to, conjugates, isomers, esters, derivatives, metabolites, residues, salts or prodrugs thereof.

Anesthetics may be, but are not limited to, local anesthetics such as procaine, benzocaine, chloroprocaine, cocaine, cyclomethycaine, dimethodcaine, larocaine, propoxycaine, proparacaine, tretracaine, lidocaine, articaine, bupivacaine, carticaine, cinchocaine, etidocaine, levobupivacaine, mepivacaine, piperocaine, prilocaine, ropivacaine, and trimecaine. A suitable concentration for the anesthetic is from about 0.01% by weight to about 6% by weight based on the total weight of the composition.

Analgesics may be, but are not limited to, paracetamol, ibuprofen, diclofenac, naproxen, aspirin, celecoxib, etoricoxib, lumiracoxib, parecoxib, rofecoxib, valdecoxib, nimesulid, oxicams, such as piroxicam, isoxicam, tonexicam, sudoxicam, and CP-14,304; the salicylates, such as salicylic acid, aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; the fenamates, such as mefenamic, meclofenamic, flufenamic, niflumic, and tolfenamic acids; the propionic acid derivates, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and the pyrazoles, such as phenybutazone, oxyphenbutazone, feprazone, azapropazone, and trimethazone.

Antimicrobials may be, but are not limited to, antibiotics such as amikacin, gentamycin, neomycin, tobramycin, kanamycin, meropenem, imipenem or cefaclor, antivirals such as abacavir, aciclovir, amantadine, boceprevir, cidofovir, darunavir, edoxudine, famciclovir, ganciclovir, imunovir, inosine, interferon, lamivudine, nexavir, oseltamivir, penciclovir, ribavirin, rimantadine, viramidine and zidovudine, and antifungals such as miconazole, ketoconazole, itraconazole, clotrimazole, econazole, fluconazole, voriconazole, abafungin, naftifine, caspofungin, micafungin, benzoic acid, and griseofulvin.

Anti-inflammatory drugs may be, but are not limited to, zinc salts, including zinc salts of polysaccharide acids, such as hyaluronic acid.

The compositions of this invention may be made into a wide variety of product types suitable for injectable administering to the target tissues that include but are not limited to solutions, gels, emulsions, suspension, microemulsions, nanoemulsions, liquid drops, liposomes, slow-releasing materials, polymers or monomers and polymerizing agents, and the like. The composition useful in the present invention can be formulated as solution. The preparation of a composition of the present invention can for instance be such that peroxisome proliferator-activated receptor-gamma is mixed in water with physiologically acceptable salts and/or thickener, like hyaluronic acid, hydroxyethylcellulose, carboxy-methyl-cellulose, glycerine or other. Such composition may further contain organic solvent.

The composition can be brought forward by any known form of preparation of aqueous mixtures.

According to an embodiment of the present invention the composition can be used for improving imperfections of the skin, for use in facial or body contouring, in facial or body shaping, as face or body filler, or for the treatment of large area volume deficiencies.

The term “large area volume deficiencies” refers to areal defects of the skin which can be larger as a single wrinkle for example facial areas as sunken cheeks or body areas as décolleté, breast, buttocks, upper arm and the like.

According to an embodiment of the invention, the imperfections of the skin are conditions or defects of the skin selected from the group consisting of wrinkled skin, furrowed skin, folding skin, sagging skin, crow's feet, scarred skin, and depressions in the skin. The imperfections of the skin can be deficiencies which are caused by e.g. ageing, environmental influences, weight loss, pregnancy, surgical interventions and acne. The imperfections of the skin especially are conditions or defects of the aging skin. The composition for example is usable to reduce the depth of skin folds, to reduce wrinkles, to fill tissue defects, and to reduce the visibility of scars. The composition in particular is suitable for treatment of wrinkles e.g. of forehead wrinkles, anger wrinkles, worry wrinkles, frown lines, medium depth wrinkles, such as nasolabial folds, glabellar lines, obvious mild to moderate nasal furrows and cheek wrinkles, perioral wrinkles, slack eyelids, crows' feet, and acne scars. The composition also is suitable for under-injection of lips and for treatment of wrinkles in the hand region, décolleté and skin indentations.

The present invention also provides an injectable composition comprising peroxisome proliferator-activated receptor-gamma for subcutaneous administration, wherein the composition comprises: a) peroxisome proliferator-activated receptor-gamma, b) a pharmaceutically acceptable carrier, and optionally c) a dermal filling material.

In the composition according to this aspect of the invention one or more pharmaceutically acceptable carriers may be present. Suitable carriers include, but are not limited to, sterile water, phosphate-buffered saline (PBS), ringer solution, isotonic saline solution (0.9%), trometamol, citrate, carbonate, acetate, borate, amino acid, diethylamine, glucono delta lactone, glycine, lactate, maleic acid, methanesulfonic acid, monoethanolamine, tartrate buffer of choice or any combination thereof.

Optionally, the composition further comprises a dermal filling material. The dermal filling material includes, but is not limited to collagen, cross-linked collagen, hyaluronic acid, poly lactic acid, calcium hydroxyl apatite, chondroitin sulfate, polyesters, polyethylene glycols, polycarbonates, polyvinyl alcohols, polyacrylamides, polyamides, polyacrylates, polyetheresters, polymethacrylates, polyurethanes, polycaprolactone, polyphophazenes, polyorthoesters, polyglycolides, copolymers of lysine and lactic acid, copolymers of lysine-RGD and lactic acid, chitosan, alginates, pectin, gelatin, gellan, carrageenan, cells, stem cells, adult stem cells, embryonic stem cells, induced pluripotent cells, progenitor cells, minced tissues, autologous transplanted cells, fat or tissues, and mixtures thereof. In a preferred embodiment, the composition of the invention can be administered initially combined with a dermal filling material and thereafter followed by an administration of the composition of the invention comprising peroxisome prolifarator activated receptor-gamma without dermal filling material as maintenance.

In one embodiment, the dermal filling material can be collagen. In another embodiment, the dermal filling material can be cross-linked collagen wherein the collagen is cross-linked with one or more sugars. In alternative embodiments, the dermal filling material is alginate. In a further embodiment, the dermal filling material is cross-linked alginate, for example barium-cross-linked alginate. In a further embodiment, the dermal filling material can be cells, for example human dermal fibroblasts. In one embodiment, the dermal filling material is hyaluronic acid. Hyaluronic acid as dermal filling material can constitute from about 0.001% by weight to about 8% by weight of the final composition. In another embodiment, the dermal filling material hyaluronic acid can constitute about 1% by weight to about 4% by weight of the final composition. In alternative embodiments, the dermal filling material hyaluronic acid can constitute about 2% by weight to about 2.5% by weight of the final composition.

The present invention also relates to the use of the composition according to the invention for improving imperfections of the skin, for use in facial or body contouring, in facial or body shaping, as face or body filler, or for the treatment of large area volume deficiencies.

According to an embodiment of the invention, the imperfections of the skin are conditions or defects of the skin selected from the group consisting of scarred skin, wrinkled skin, furrowed skin, folding skin, sagging skin, crow's feet and depressions in the skin. The imperfections of the skin especially are conditions or defects of the aging skin. The composition according to the invention can be used to reduce wrinkles, to reduce the depth of skin folds, to fill tissue defects, or to reduce the visibility of scars. The composition in particular can be used for treatment of wrinkles e.g. of forehead wrinkles, anger wrinkles, worry wrinkles, frown lines, medium depth wrinkles, such as nasolabial folds, glabellar lines, obvious mild to moderate nasal furrows and cheek wrinkles, perioral wrinkles, slack eyelids, crows' feet, and acne scars. The composition also is suitable for under-injection of lips and for treatment of wrinkles in the hand region, decollete and skin indentations.

According to an embodiment of the invention, the composition of the invention can be used as dermal filler. The composition of the invention for example can be used as injectable filler to reduce the depth of skin folds, to reduce wrinkles, to fill tissue defects, or to reduce the visibility of scars. According to an embodiment, the composition of the invention can be used as a natural injectable filler for the aesthetic treatment of wrinkles.

The present invention also relates to a method for improving imperfections of the skin, wherein the injectable composition according to the present invention is subcutaneously administered at the area of skin imperfections comprising the steps: a) identifying an area of skin imperfections, b) administering a safe and cosmetically effective amount of the composition subcutaneously or dermal to the area of skin imperfections.

In one embodiment, the composition of this invention is administered by subcutaneous injection. According to another embodiment, the composition is subcutaneously or dermal administered by intradermal and/or subdermal injection.

In an embodiment, the composition is injected through a needle or other suitable techniques. The injection can be carried out either by multiple or several-fold injection into the areas of skin affected. Alternatively, the injection can be carried out one to several times. In an embodiment one injection shot is sized from about 0.15 ml of the composition to about 5 ml. In another embodiment one injection shot is sized from about 0.5 ml of the composition to about 2 ml.

The invention described here is suitable for skin imperfections which are caused by e.g. ageing, environmental influences, weight loss, pregnancy, surgical interventions and acne. The imperfections of the skin especially are conditions or defects of the aging skin. According to an embodiment of the invention, the imperfections of the skin are conditions or defects of the skin selected from the group consisting of wrinkled skin, furrowed skin, folding skin, sagging skin, crow's feet, scarred skin, and depressions in the skin. The composition in particular is suitable for treatment of wrinkles e.g. of forehead wrinkles, anger wrinkles, worry wrinkles, frown lines, medium depth wrinkles, such as nasolabial folds, glabellar lines, obvious mild to moderate nasal furrows and cheek wrinkles, perioral wrinkles, slack eyelids, crows' feet, and acne scars. The composition also is suitable for under-injection of lips and for treatment of wrinkles or volume deficiencies in the hand region and decollete and skin indentations after liposuction of cellulite or other skin areas. The method for example is usable to reduce the depth of skin folds, to reduce wrinkles, to fill tissue defects, and to reduce the visibility of scars or to improve large area volume loss. The method can improve the facial contour around said area of facial skin.

The invention will be explained in more detail by virtue of the examples set forth herein below. While at least one exemplary embodiment is presented, it should be appreciated that a vast number of variations exist. It should also be appreciated that the exemplary embodiments are only examples, and are not intended to limit the scope, applicability, or configuration in any way. Rather, the forgoing description will provide those with ordinary skill in the art with the essential characteristics of this invention for implementing at least one exemplary embodiment, it being understood that various changes may be made without departing from the scope as set forth in the appended claims.

### FIGURES

In the figures show:

FIG. 1 shows a photometric measurement of an MTT assay of adipose-derived stem cells incubated for 6 days with different concentrations of PPARγ (OD×10⁻³). The bars 1 to 10 refer to adipose-derived stem cells incubated in NM showing negative controls incubated with DMEM without FCS (1), controls incubated with standard culture medium NM (2), solvent controls incubated with NM containing DMSO (3), cells incubated with 2.7 μg/ml (4), 1 μg/ml (5) 0.5 μg/ml (6), or 0.1 μg/ml (7) of PPARγ, 5 μM Pioglitazone (8), 5 μM Pioglitazone and 2.7 μg/ml PPARγ (9), or 5 μM Pioglitazone and 0.1 μg/ml PPARγ (10) in NM. The bars 11 to 18 refer to adipose-derived stem cells incubated in AM- showing controls incubated with AM- (11), cells incubated with 2.7 μg/ml (12), 1 μg/ml (13) 0.5 μg/ml (14), or 0.1 μg/ml (15) of PPARγ, 5 μM Pioglitazone (16), 5 μM Pioglitazone and 2.7 μg/ml PPARγ (17), or 5 μM Pioglitazone and 0.1 μg/ml PPARγ (18) in AM-.

FIG. 2 shows a photometric measurement of cell cultures of adipose-derived stem cells incubated for 2 weeks with different concentrations of PPARγ. The bars 1 to 8 refer to adipose-derived stem cells incubated in NM showing controls incubated with standard culture medium NM (1), and cells incubated with 2.7 μg/ml (2), 1 μg/ml (3) 0.5 μg/ml (4), or 0.1 μg/ml (5) of PPARγ, 5 μM Pioglitazone (6), 5 μM Pioglitazone and 2.7 μg/ml PPARγ (7), or 5 μM Pioglitazone and 0.1 μg/ml PPARγ (8) in NM. The bars 9 to 16 refer to adipose-derived stem cells incubated in AM- showing controls incubated with AM- (9), cells incubated with 2.7 μg/ml (10), 1 μg/ml (11) 0.5 μg/ml (12), or 0.1 μg/ml (13) of PPARγ, 5 μM Pioglitazone (14), 5 μM Pioglitazone and 2.7 μg/ml PPARγ (15), or 5 μM Pioglitazone and 0.1 μg/ml PPARγ (16) in AM-.

FIG. 3 (magnification of 630×) shows an Oil-O-Red staining of adipose-derived stem cells incubated in standard culture medium (NM) with different concentrations of PPARΓ. FIG. 3a) shows control cells, FIGS. 3b), 3c), 3d), and 3e) show cells cultured with 2.7 μg/ml, 1 μg/ml, 0.5 μg/ml, or 0.1 μg/ml of PPARγ, respectively.

### EXAMPLES

### Example 1

### Preparation Of Peroxisome Proliferator-Activated Receptor-Gamma Protein

For expression of peroxisome proliferator-activated receptor-gamma protein in B. megaterium, the procedure was carried out analogously to: Barg, H., Malten, M. & Jahn, D. (2005) “Protein and vitamin production in Bacillus megaterium”, Methods in Biotechnology-Microbial Products and Biotransformations (Barredo, J.-L., ed.). As fungal production strains can be used Pichia pastoris (for example GS115 and KM71 (Invitrogen) and others) and Aspergillus nidulans (for example RMS011 (Stringer, M A, Dean, R A, Sewall, T C, Timberlake, W E (1991) “Rodletless, a new Aspergillus developmental mutant induced by direct gene activation”, Genes Dev 5:1161-1171) and SRF200 (Karos, M, Fischer, R (1999) “Molecular characterization of HymA, an evolutionarily highly conserved and highly expressed protein of Aspergillus nidulans”, Mol Genet Genomics 260:510-521), and others). Further, it is also possible to use other fungal production hosts, such as, for example, Aspergillus niger for the expression. Alternatively, peroxisome proliferator-activated receptor-gamma protein can be produced recombinantly in E. coli, for example using vector systems such as pQE30. Purification can for example be effected by affinity chromatography (for example His-Tag).

### Example 2

### Induction of Adipocyte Differentiation by Peroxisome Proliferator-Activated Receptor-Gamma

The ability of peroxisome proliferator-activated receptor-gamma to induce differentiation of adipocytes was studied in adipose-derived stem cells (ASC). Human adipose-derived stem cells were obtained by liposuction and cultured following a published procedure (Crandall et al, Endocrinology 140:154-8, 1999). In brief, adipose tissue was digested with 2 mg/mL collagenase in Krebs-Ringer-bicarbonate buffer (pH 7.4). The preadipocyte fraction was resuspended in growth medium, transferred to a culture flask, and maintained in an incubator at 37° C., and 5% CO2. Cell attachment was allowed for 16-20 h and afterwards the cells that had not adhered were removed.

Peroxisome proliferator-activated receptor-gamma (PPARgamma) was added to respective final concentrations of 0.0001%, 0.01% and 1% by weight to the culture media 24 hours after the cells were seeded. The media was changed every three days and supplemented with fresh peroxisome proliferator-activated receptor-gamma. The cells were incubated with peroxisome proliferator-activated receptor-gamma for 5 days. Afterwards peroxisome proliferator-activated receptor-gamma was removed, and the cells were kept for further 9 days in culture for observation of lipid droplet formation, which is indicative of the ability to differentiate. Controls were run in medium without peroxisome proliferator-activated receptor-gamma. The formation of lipid droplets was verified by oil red O staining, a fat-soluble dye used for staining of neutral triglycerides in chamber slides by microscope and 96-well flat-bottom microplates by spectrophotometry (540 nm). Increased oil red O staining correlated with more lipid production, and therefore with higher level of differentiation. The results are given in the table 1 below:

TABLE 1

| Treatment | Degree of differentiation |
|---|---|
| medium | − to + |
| medium and PPARgamma | ++ to +++ |

− no differentiation
+ minimal differentiation
++ moderate differentiation
+++ high differentiation

Table 1 shows that adipocytes show increased differentiation upon treatment with peroxisome proliferator-activated receptor-gamma, relative to controls. This example suggests that treatment of adipocytes with peroxisome proliferator-activated receptor-gamma in vivo will improve imperfections of the skin.

### Example 3

### Isolation of Human Adipose-Derived Stem Cells

Human adipose-derived adult mesenchymal stem cells (ASCs) were isolated from lipoaspirates from patients undergoing cosmetic liposuction. Liposuction aspirates were obtained from patients undergoing plastic surgical procedures. Aspirated tissue was digested at 37° C. with 0.075% collagenase I (230 U/mg; CellSystems, Germany) and continuously agitated for 45 minutes. The stromalvascular fraction was separated from the remaining fibrous material and the floating adipocytes by centrifugation at 300 g. The sedimented cells were washed with PBS (Thermo Scientific-Pierce, Germany) and filtered through a 100 μm pore filter (Millipore, Germany). Erythrocyte contamination was reduced by density gradient centrifugation with Biocoll (Biochrom, Germany) as high contamination with erythrocytes was found to markedly decrease cell adherence and proliferation. Finally, the cells were plated for initial cell culture, and cultured at 37° C. in an atmosphere of 5% CO₂ in humid air. Dulbecco's modified Eagle's medium (DMEM, Sigma, Germany) with a physiologic glucose concentration of 100 mg/dl supplemented with 10% fetal calf serum (FCS; PAA, Germany) was used as the culture medium. The medium was replaced every three days. Subconfluent cells were passaged by trypsinization.

### Example 4

### Determination of the Effect of PPARγ on the Cell Proliferation of Adipose-Derived Stem Cells

Cell proliferation was determined by a photometric assay using MTT (3-(4,5-dimethylthiazol-2-yl)-2,5-diphenyltetrazolium bromide). In brief, human adipose-derived stem cells were isolated from 5 different donors according to example 3 and 1.5×10⁴ cells were seeded in 96-well-plates. Cell samples were used in quadruplicate in each experiment. The human adipose-derived stem cells were cultured for 6 days in two different media containing PPARγ or control media. Recombinant PPARγ (recPPARγ) (Thermo Scientific-Pierce, PPARgamma human) was solved in dimethyl sulfoxide (DMSO) at a concentration of 270 g/ml and used as a stock solution. Working solutions were prepared by dilution using a factor of 1:100 for a final concentration of 2.7 μg/ml, a factor of 1:270 for a final concentration of 1 μg/ml, a factor of 1:540 for a final concentration of 0.5 μg/ml, and a factor of 1:2700 for a final concentration of 0.1 μg/ml.

Cell samples were cultured in Dulbecco's modified Eagle's medium with a physiologic glucose concentration of 100 mg/dl (DMEM, Sigma, Germany) supplemented with 10% fetal calf serum (FCS; PAA, Germany), which is a standard culture medium denoted “NM”, containing 2.7 μg/ml, 1 μg/ml, 0.5 μg/ml, or 0.1 μg/ml of PPARγ, respectively. Further samples were cultured in NM containing 5 μM Pioglitazone (Sigma-Aldrich, Germany), or 5 μM Pioglitazone and 2.7 μg/ml PPARγ, or 5 μM Pioglitazone and 0.1 μg/ml PPARγ. Control cells were cultured in NM. Cell samples incubated with DMEM without FCS were used as a negative control. Solvent controls were incubated with NM containing DMSO. Other cell samples were cultured in DMEM with 4500 mg/l glucose (high glucose) and 10 μM insulin (Novo Nordisk), 1 μM dexamethasone (Ratiopharm), and 10% FCS, which is denoted “AM-”. Cell samples were cultured in AM- containing 2.7 μg/ml, 1 μg/ml, 0.5 μg/ml, or 0.1 μg/ml of PPARγ, respectively, or 5 μM Pioglitazone, or 5 μM Pioglitazone and 2.7 μg/ml PPARγ, or 5 μM Pioglitazone and 0.1 μg/ml PPARγ. Control cells were cultured in AM-.

After 6 days of culturing, the cells were incubated in PBS containing MTT at 37° C. for 4 hours. Afterwards, the medium was removed, 0.04 N HCl was added, and absorbance was measured in a microplate reader at 550 nm versus 650 nm.

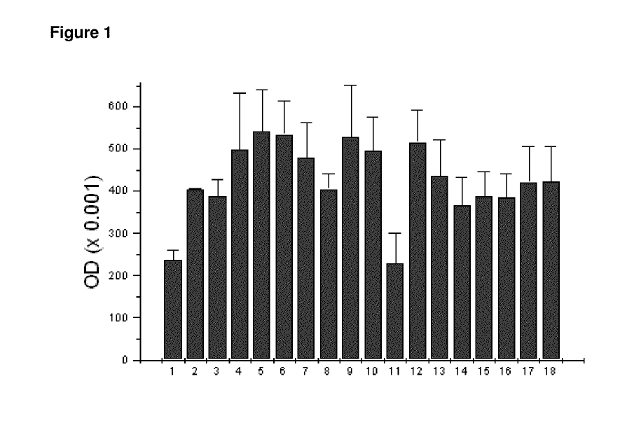

FIG. 1 illustrates the effect of PPARγ on the cell proliferation showing the photometric measurement (OD×10⁻³) of the cell samples. In FIG. 1, bars 1 to 10 refer to adipose-derived stem cells incubated in NM showing negative controls incubated with DMEM without FCS (1), controls incubated with standard culture medium NM (2), solvent controls incubated with NM containing DMSO (3), cells incubated with 2.7 μg/ml (4), 1 μg/ml (5) 0.5 μg/ml (6), or 0.1 μg/ml (7) of PPARγ, 5 μM Pioglitazone (8), 5 μM Pioglitazone and 2.7 μg/ml PPARγ (9), or 5 μM Pioglitazone and 0.1 μg/ml PPARγ (10) in NM. The bars 11 to 18 refer to adipose-derived stem cells incubated in AM- showing controls incubated with AM- (11), cells incubated with 2.7 μg/ml (12), 1 μg/ml (13) 0.5 μg/ml (14), or 0.1 μg/ml (15) of PPARγ, 5 μM Pioglitazone (16), 5 μM Pioglitazone and 2.7 μg/ml PPARγ (17), or 5 μM Pioglitazone and 0.1 μg/ml PPARγ (18) in AM-.

As can be taken from FIG. 1, the results of the MTT assay illustrated that PPARγ stimulated the proliferation of the adipose-derived stem cells in a dose-dependent manner in both media. Pioglitazone alone had no effect on cell proliferation in NM standard medium.

### Example 5

### Photometric Measurement of Adipogenic Differentiation in Adipose-Derived Stem Cells Induced by PPARγ

The induction of adipogenic differentiation by recombinant PPARγ was tested in human adipose-derived stem cells from 4 different donors isolated as described in example 3. The cells were cultured for 2 weeks, and media were changed every 5-6 days. Cell samples were seeded with 1.5×10⁴ cells in 96-well-plates. Recombinant PPARγ (recPPARγ) (Thermo Scientific-Pierce, PPARgamma human) was solved in dimethyl sulfoxide (DMSO) at a concentration of 270 g/ml and used as a stock solution. Working solutions were prepared by dilution using a factor of 1:100 for a final concentration of 2.7 μg/ml, a factor of 1:270 for a final concentration of 1 μg/ml, a factor of 1:540 for a final concentration of 0.5 μg/ml, and a factor of 1:2700 for a final concentration of 0.1 μg/ml.

Cells were cultured in two different media, either in “NM”, a standard culture medium of Dulbecco's modified Eagle's medium with a physiologic glucose concentration of 100 mg/dl (DMEM, Sigma, Germany) supplemented with 10% fetal calf serum (FCS; PAA, Germany), or in “AM-” composed of DMEM with 4500 mg/l glucose (high glucose) and 10 μM insulin (Novo Nordisk), 1 μM dexamethasone (Ratiopharm), and 10% FCS. Cell samples were cultured in NM containing 2.7 μg/ml, 1 μg/ml, 0.5 μg/ml, or 0.1 μg/ml of PPARγ, respectively. Further samples were cultured in NM containing 5 μM Pioglitazone (Sigma-Aldrich, Germany), or 5 μM Pioglitazone and 2.7 μg/ml PPARγ, or 5 μM Pioglitazone and 0.1 μg/ml PPARγ. Control cells were cultured in NM. Further cell samples were cultured in AM- containing 2.7 μg/ml, 1 μg/ml, 0.5 μg/ml, or 0.1 μg/ml of PPARγ, respectively, or 5 μM Pioglitazone, or 5 μM Pioglitazone and 2.7 μg/ml PPARγ, or 5 μM Pioglitazone and 0.1 μg/ml PPARγ. Control cells were cultured in AM-. After 2 weeks of culturing, the optical density was measured in a microplate reader.

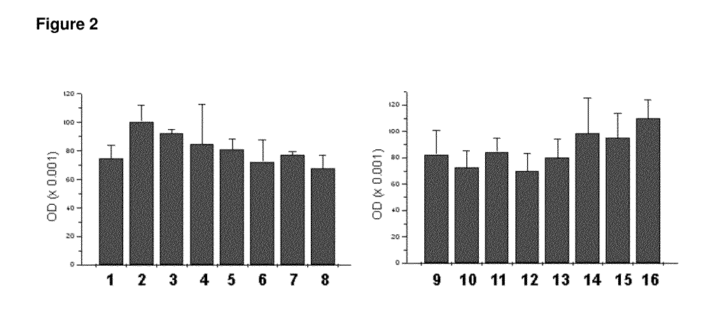

FIG. 2 shows the optical density (OD×10⁻³) of the cell samples after 2 weeks of culturing. In FIG. 2, bars 1 to 8 refer to adipose-derived stem cells incubated in NM showing controls incubated with standard culture medium NM (1), and cells incubated with 2.7 μg/ml (2), 1 μg/ml (3) 0.5 μg/ml (4), or 0.1 μg/ml (5) of PPARγ, 5 μM Pioglitazone (6), 5 μM Pioglitazone and 2.7 μg/ml PPARγ (7), or 5 μM Pioglitazone and 0.1 μg/ml PPARγ (8) in NM. The bars 9 to 16 refer to adipose-derived stem cells incubated in AM- showing controls incubated with AM- (9), cells incubated with 2.7 μg/ml (10), 1 μg/ml (11) 0.5 μg/ml (12), or 0.1 μg/ml (13) of PPARγ, 5 μM Pioglitazone (14), 5 μM Pioglitazone and 2.7 μg/ml PPARγ (15), or 5 μM Pioglitazone and 0.1 μg/ml PPARγ (16) in AM-.

As can be taken from FIG. 2, the measurement of optical density illustrated that incubation with PPARγ did not reduce the cell number. This showed that PPARγ had no cytotoxic effect on cells. Further, the increase in optical density shows that the incubation with PPARγ stimulated the differentiation and proliferation of the adipose-derived stem cells in both media.

### Example 6

### Induction of Differentiation of Adipose-Derived Stem Cells by Recombinant PPARγ

The induction of adipogenic differentiation by recombinant PPARγ was verified by Oil-O-Red staining of fat vacuoles. Human adipose-derived stem cells (ASCs) from 4 different donors were isolated as described in example 3. The cells were cultured for 2 weeks in “NM”, a standard culture medium of Dulbecco's modified Eagle's medium with a physiologic glucose concentration of 100 mg/dl (DMEM, Sigma, Germany) supplemented with 10% fetal calf serum (FCS; PAA, Germany) and medium was changed every 5-6 days. Cells were cultured in NM containing 2.7 μg/ml, 1 μg/ml, 0.5 μg/ml, or 0.1 μg/ml of PPARγ (Thermo Scientific-Pierce, PPARgamma human), respectively, or 5 μM Pioglitazone (Sigma-Aldrich, Germany), or 5 μM Pioglitazone and 2.7 μg/ml PPARγ, or 5 μM Pioglitazone and 0.1 μg/ml PPARγ. Controls were cultured in NM.

After 2 weeks in culture the formation of lipid droplets which is indicative of the ability to differentiate was verified by Oil-O-Red staining. Oil-O-Red is a fat-soluble dye used for the staining of neutral triglycerides. Oil-O-Red staining reveals the accumulation of lipid droplets in intracellular vacuoles indicating adipogenic differentiation. After fixation with 4% paraformaldehyde for 20 minutes, the cells were incubated for a staining time of 1 hour with a 0.5% Oil-O-Red (Sigma) solution in Isopropanol/glycerine. The Oil-O-Red staining was evaluated microscopically.

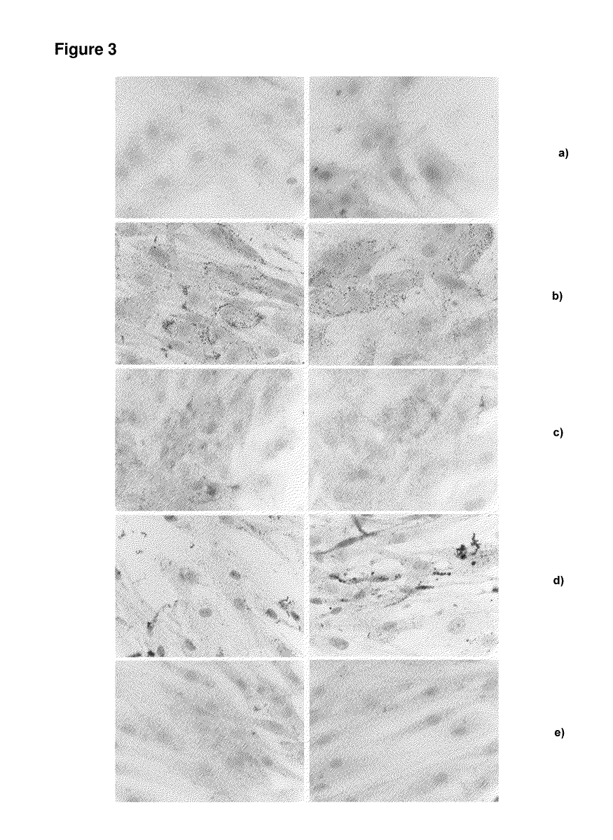

The FIG. 3 shows the effect of PPARγ on the differentiation of adipose-derived stem cells after 2 weeks of culture. FIG. 3a) shows microscopic pictures (magnification: 630×) of undifferentiated control cells cultured in NM. FIGS. 3b), 3c), 3d), and 3e) show microscopic pictures of cells cultured for 2 weeks with 2.7 μg/ml, 1 μg/ml, 0.5 μg/ml, or 0.1 μg/ml of PPARγ, respectively. As can be taken from FIGS. 3b), 3c), 3d), and 3e), the Oil-O-Red staining showed that PPARγ was able to induce the formation of fat vacuoles and hence adipogenic differentiation of adipose-derived stem cells. Further the Figures show that the induction of formation of fat vacuoles by PPARγ was a concentration dependent effect.

Further cells were cultured in NM containing 5 μM Pioglitazone (Sigma-Aldrich, Germany), or 5 μM Pioglitazone and 2.7 μg/ml PPARγ, or 5 μM Pioglitazone and 0.1 μg/ml PPARγ. It was detected that the combination of PPARγ plus Pioglitazone further increased the development of fat vacuoles compared with pure NM medium.

Further cell samples were cultured in “AM-” composed of DMEM with 4500 mg/l glucose, 10 μM insulin (Novo Nordisk), 1 μM dexamethasone (Ratiopharm), 10% FCS. Cell samples were cultured in AM- containing 2.7 μg/ml, 1 μg/ml, 0.5 μg/ml, or 0.1 μg/ml of PPARγ, respectively, or 5 μM Pioglitazone, or 5 μM Pioglitazone and 2.7 μg/ml PPARγ. Negative control cells were cultured in AM-. Cells incubated with DMEM with 4500mg/l glucose, 10 μM insulin (Novo Nordisk), 1 μM dexamethasone (Ratiopharm), 10% FCS and 1 mM isobutyl-methylxanthin (Sigma) and 200 μM indometacin (Fluka) were used as a positive induction control. It was detected that also in AM- medium PPARγ was able to induce the formation of fat vacuoles and hence adipogenic differentiation of adipose-derived stem cells in a concentration-dependent manner.

Culturing of cells in NM and in AM- containing the different PPARγ concentrations as given above was repeated with an incubation period of 3 weeks. The Oil-O-Red staining revealed that after 3 weeks of incubation in NM PPARγ heavily induced adipogenic differentiation. While the addition of 5 μM Pioglitazone only slightly induced differentiation, the combination of 2.7 μg/ml PPARγ and 5 μM Pioglitazone massively induced the development of fat vacuoles. Further, it was detected that also in cells cultured for 3 weeks in AM- medium PPARγ induced the formation of fat vacuoles and adipogenic differentiation of adipose-derived stem cells in a concentration-dependent manner.

These experiments show that PPARγ is able to induce adipogenic differentiation in adipose-derived stem cells and suggest that treatment of adipocytes with peroxisome proliferator-activated receptor-gamma in vivo will improve imperfections of the skin.

1-15. (canceled)

16. An injectable composition comprising peroxisome proliferator-activated receptor-gamma.

17. The composition of claim 16, wherein the composition comprises at least one agonist of the peroxisome proliferator-activated receptor-gamma selected from the group consisting of eicosanoids especially prostaglandins such as Δ12-prostaglandin J2 and 15-deoxy-Δ12-prostaglandin J2, thiazoliden derivatives such as rosiglitazone, ciglitazone, troglitazone, englitazone and pioglitazone, non steroidal anti-inflammatory drugs, unsatturated fatty acids, alpha-linoleic acid, arachidonic acid, docosahexaenoic acid, eicosapentaenoic acid, biphenyl derivatives, N-(phenyloxazol-4-yl-methoxymethyl)-cyclohexyl-succinic acid amide derivatives, and mixtures thereof.

18. The composition of claim 16, wherein the composition comprises retinoic acid, retinol, retinal and/or retinoid X receptor.

19. The composition of claim 16, wherein the peroxisome proliferator-activated receptor-gamma has a concentration in the range from about 0.00001% by weight to about 5% by weight, based on the total weight of the composition.

20. The composition of claim 17, wherein the agonist of the peroxisome proliferator-activated receptor-gamma has a concentration in the range from about 0.00001% by weight to about 5% by weight, based on the total weight of the composition.

21. The composition of claim 18, wherein the retinoic acid, retinol, retinal or retinoid X receptor has a concentration in the range from about 0.00001% by weight to about 5% by weight, based on the total weight of the composition.

22. The composition of claim 16, wherein the composition further comprises a dermal filling material selected from the group consisting of collagen, cross-linked collagen, hyaluronic acid, crosslinked hyaluronic acid, poly lactic acid, calcium hydroxyl apatite, chondroitin sulfate, polyesters, polyethylene glycols, polycarbonates, polyvinyl alcohols, polyacrylamides, polyamides, polyacrylates, polyetheresters, polymethacrylates, polyurethanes, polycaprolactone, polyphophazenes, polyorthoesters, polyglycolides, copolymers of lysine and lactic acid, copolymers of lysine-RGD and lactic acid, chitosan, alginates, pectin, gelatin, gellan, carrageenan, cells, stem cells, adult stem cells, embryonic stem cells, induced pluripotent cells, progenitor cells, minced tissues, autologous transplanted cells, fat or tissues, and mixtures thereof.

23. The composition of claim wherein the c position is for subcutaneous injection.

24. The composition of claim 16, for improving skin imperfections, for use in facial or body contouring, in facial or body shaping, as a face or body filler, or for the treatment of large area volume deficiencies.

25. The composition of claim 24, wherein the skin imperfections are conditions or defects selected from the group consisting of wrinkled skin, furrowed skin, folding skin, sagging skin, crow's feet, scarred skin and, depressions.

26. An injectable composition comprising peroxisome proliferator-activated receptor-gamma for subcutaneous administration, wherein the composition comprises: a) peroxisome proliferator-activated receptor-gamma, b) a pharmaceutically acceptable carrier, and optionally c) a dermal filling material.

27. A method for improving imperfections of the skin, for use in facial or body contouring, in facial or body shaping, as face or body filler, or for the treatment of large area volume deficiencies, comprising administering a composition of claim 16.

28. The method of claim 27 for use as dermal filler.

29. A method for improving imperfections of the skin, comprising the steps; a) identifying an area of skin imperfections, b) administering a safe and cosmetically effective amount of a composition of claim 16 subcutaneously or dermal to the area of skin imperfections.

30. The method of claim 29, wherein the composition is subcutaneously or dermal administered by intradermal and/or subdermal injection.
